(19) United States
(12) Patent Application Publication
(10) Pub. No.: US 2008/0257719 A1
(43) Pub. Date: Oct. 23, 2008

(54) Apparatus And Method For Making Flammable Gas

(76) Inventor: Ted Suratt, (US)

Correspondence Address: Eugenia S. Hansen;Hemingway & Hansen, LLP, Comerica Bank Tower Suite # 2500, 1717 Main Street, Dallas, TX, 75201, US

(21) Appl. No.: 11/738,476

(22) Filed: Apr. 21, 2007

(51) Int. Cl. C25B 9/00

(52) U.S. Cl. 204/272

(57) ABSTRACT

An electrolyzer includes a first electrode in the form of an inner tank formed of a material capable of being magnetized and a second electrode in the form of an outer tank formed of a material capable of being magnetized. The inner tank is nested within the outer tank. A spacer basket formed of a plastic material is disposed between the inner tank and the outer tank. The inner tank includes a positive terminal in electrical communication with a DC power source and the outer tank includes a negative terminal in electrical communication with the DC power source. A magnet for magnetizing the outer tank has a field sufficient to affect the inner tank. Electrolysis within the tank produces a flammable gas having a first hydrogen atom bonded to an oxygen atom and a second hydrogen atom bonded to the first hydrogen atom.

### BACKGROUND OF THE INVENTION

1. Field of the Invention

This invention relates, generally, to the generation of gas from electrolysis. More particularly, it relates to the generation of a flammable gas having unusual properties and a heretofore unknown molecular structure.

2. Description of the Prior Art

It has long been known that water can be separated into hydrogen and oxygen with the use of an electrolyzer. However, the cost of the process is greater than the benefits received.

What is needed, then, is an improved electrolysis method and apparatus that produces a novel flammable gas from an electrolyte and water solution that provides benefits without the prior art high cost of separation.

However, in view of the prior art considered as a whole at the time the present invention was made, it was not obvious to those of ordinary skill in the pertinent art how the identified needs could be fulfilled.

### SUMMARY OF THE INVENTION

The long-standing but heretofore unfulfilled need for a novel flammable gas and for an electrolyzer capable of generating such novel gas is now provided in the form of a new, useful, and nonobvious invention.

Because molecules such as hydrogen and oxygen have magnetic qualities, they exist normally in a diatomic state O₂ and H₂. They also have electronegative values that affect the strength of the bond. In the case of a water molecule (H₂O), this invention discloses that introducing a current through an electrolyte solution, preferably a potassium hydroxide solution, in the presence of a magnetic field reconfigures the molecule into a heretofore unknown gas referred to hereinafter as hydroxyhydrogen or hydrogas. This heretofore unknown gas is a diatomic hydrogen molecule that is single bonded to atomic oxygen.

This is accomplished by spacing an anode and a cathode about a quarter of an inch apart and introducing a DC voltage of approximately 1.5 to 2.0 volts with current equal to approximately one-quarter of an amp (0.25 amp) per square inch of cathode surface area. This weakens the electronegative strength of the oxygen atom and allows a hydrogen atom to dislodge and magnetically bond to the other hydrogen atom that is strengthened by the magnetic field.

The resulting product is a flammable non-toxic gas that implodes when lit, and is an oxidizer that liberates atomic hydrogen when it bonds with another molecule.

More particularly, the novel electrolyzer includes a first electrode in the form of an inner tank having a first diameter formed of a material capable of being magnetized and a second electrode in the form of an outer tank having a second diameter formed of a material capable of being magnetized. The second diameter is greater than the first diameter and the inner tank is nested within the outer tank.

A cylindrical open space is created between the inner and outer tanks by the difference in tank diameters. Drain holes are formed in the sidewalls of the inner tank, near the bottom end thereof, so that when the inner tank is filled water and an electrolyte such as potassium hydroxide to form an electrolytic fluid, the electrolytic liquid fluid flows through the drain holes and is disposed in the open space between the inner tank and said outer tank. The inner tank is also full of said electrolytic fluid when said open space is full.

A spacer basket formed of a plastic material is disposed in the open space between the inner tank and the outer tank.

The positive side of a DC power source is in electrical communication with a positive terminal secured to the inner tank and the negative side of the DC power source is in electrical communication with a negative terminal secured to the outer tank. The DC power source provides a voltage of about 1.5 to 2.0 volts and a current of about 0.25 amps per square inch of cathode surface area.

A magnet having a strength of about twenty (20) Gauss units magnetizes the outer tank, and the strength of the magnetic field of the magnet is sufficient to affect the inner tank.

The outer tank has a gas outlet and a flammable gas is generated by electrolysis within the electrolyzer and is collected as it exits through said gas outlet. The flammable gas burns at a temperature of about two hundred seventy degrees Fahrenheit (270° F.) when ignited. The flammable gas has a first hydrogen atom bonded with an oxygen atom and a second hydrogen atom bonded with the first hydrogen atom.

A purified, polarized water is made by passing the flammable gas through distilled water.

An important object of this invention is to provide a method and apparatus for making the novel gas.

These and other important objects, advantages, and features of the invention will become clear as this description proceeds.

The invention accordingly comprises the features of construction, combination of elements, and arrangement of parts that will be exemplified in the description set forth hereinafter and the scope of the invention will be indicated in the claims.

### BRIEF DESCRIPTION OF THE DRAWINGS

For a fuller understanding of the nature and objects of the invention, reference should be made to the following detailed description, taken in connection with the accompanying drawings, in which:

FIG. 1 is a prior art diagram of a water molecule;

FIG. 2 is a diagram of the novel molecule;

FIG. 3 is a diagram of the novel electrolyzer;

FIG. 4 is a is a diagram of the novel spacer basket; and

FIG. 5 is a diagram of the magnetized outer tank of the novel electrolyzer.

### DETAILED DESCRIPTION OF THE PREFERRED EMBODIMENT

The novel electrolyzer is surrounded by a magnetic field that may be generated by an electromagnet or a permanent magnet. Carefully controlled voltage and amperage reconfigures a water molecule into a magnecule, referred to hereinafter as hydroxyhydrogen.

More particularly, a current is passed through a conductive water solution. The current weakens the bonding strength of the water and thus allows the magnetic force provided by the magnetic field to reconfigure the water molecule into a highly polar magnacule containing atomic oxygen bonded to a diatomic molecule of hydrogen. The resulting product is a flammable gas. When lit by an ignition source, the hydroxyhydrogen gas implodes and produces a very low temperature flame of approximately two hundred seventy degrees Fahrenheit (270° F.).

The total heat energy is approximately equal to the power required to create the gas.

Due to its low temperature, the gas has limited utility as a stand alone fuel. However, when the flame is applied to a substrate such as iron, the flame temperature increases almost immediately to the melting temperature of iron. The same phenomena holds true for metals other than iron and substances other than metals; the flame temperature rises quickly to the melting temperature of the metal or other substance.

The electronegative value of iron is lower than the electronegative value of hydrogen. This causes the highly energized hydroxyl molecule to attach itself to the iron and release hydrogen. The released hydrogen bonds with atmospheric oxygen and thereby creates large amounts of heat. This quality makes the gas highly useful in the metal-cutting industry and reduces the amount of hydrocarbons used and thus reduces the amount of byproducts of hydrocarbons that are released into the atmosphere.

Although the novel gas is not a fuel, when added to the air intake of a combustion engine, the novel gas dramatically reduces harmful exhaust emissions and increases fuel efficiency. This reduces oil consumption. It is a safe, non-toxic oxidizer that bonds easily with water.

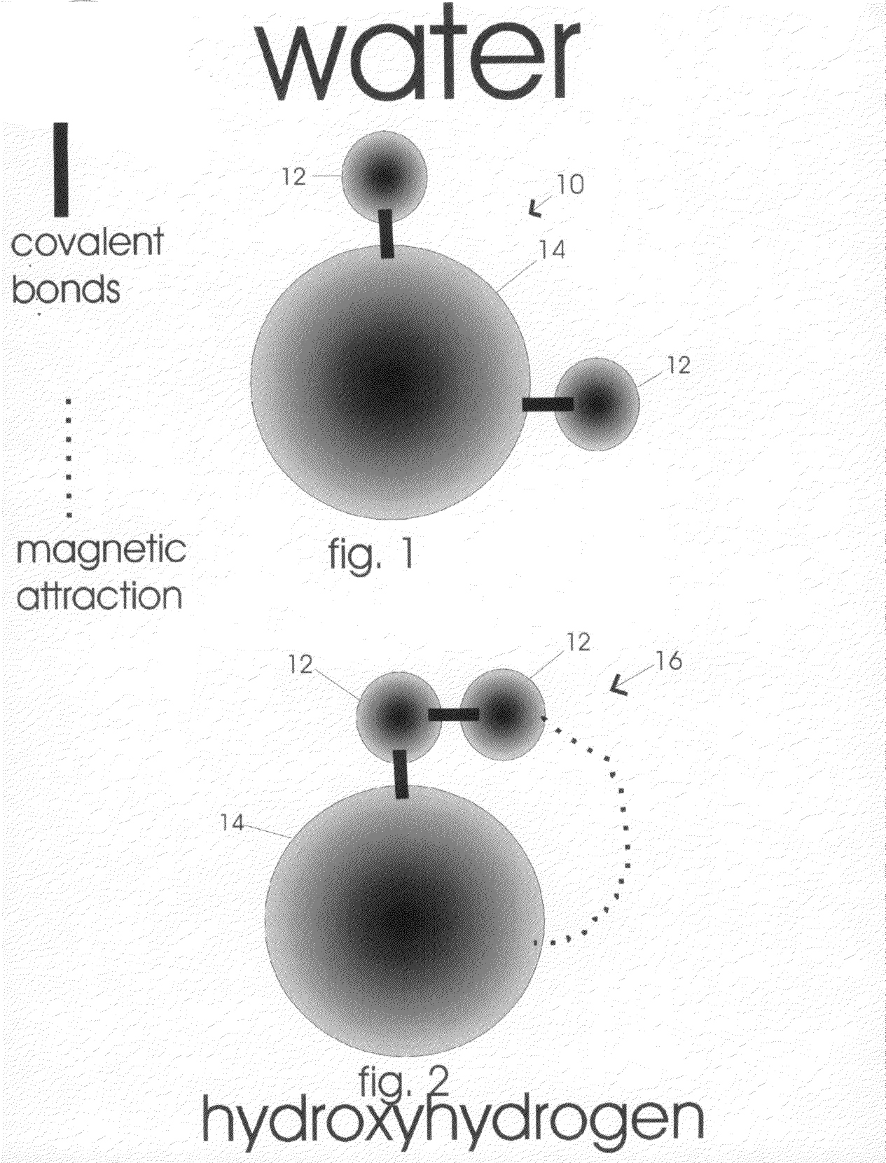

Referring now to FIG. 1, it will there be seen that a water molecule is denoted 10 as a whole. As is well-known, it includes two (2) hydrogen atoms, collectively denoted 12, and one (1) oxygen atom, denoted 14.

A hydroxyhydrogen magnecule 16 is disclosed in FIG. 2. It also includes two (2) hydrogen atoms 12 and one (1) oxygen atom 14. However, a first hydrogen atom has been released from its bond with oxygen atom 14 and has bonded with a second hydrogen atom as depicted.

Conventional science maintains that hydrogen can have but one (1) bond, as depicted in FIG. 1. It follows that the hydrogen atom 12 in FIG. 2 that seems to have two (2) bonds (one with oxygen atom 14 and one with another hydrogen atom) cannot exist in nature. However, the properties and behavior of the novel gas may be explained if the structure depicted in FIG. 2 is correct.

Ionic and covalent bonding could better be described as electrostatic and magnetic bonding, respectively. The Lewis dot theory of bonding which is taught in all universities is flawed in many ways. Lewis himself recognized that nitrogen, which can have a valence of 3, 4, or 5, was an exception to the rule. Diatomic molecules such as hydrogen are also exceptions. Based on the Lewis dot theory, hydrogen can have only one bond. This contradicts Gauss's Law which states that there are no monopoles in magnetism, i.e., there are only dipoles. Water is proof that Gauss is correct since it is well known that liquid water forms hydrogen bonds with other water molecules in order to remain in a liquid solution. Individual H₂O molecules from evaporation or transpiration are a gas. Based on the atomic weight of the molecule, water is a gas. By applying a small amount of energy in an electrolyzer, we can separate the water molecules into a gaseous form and with the presence of a magnetic field re-establish the diatomic hydrogen bond. The magnetic strength should be approximately twenty (20) Gauss units.

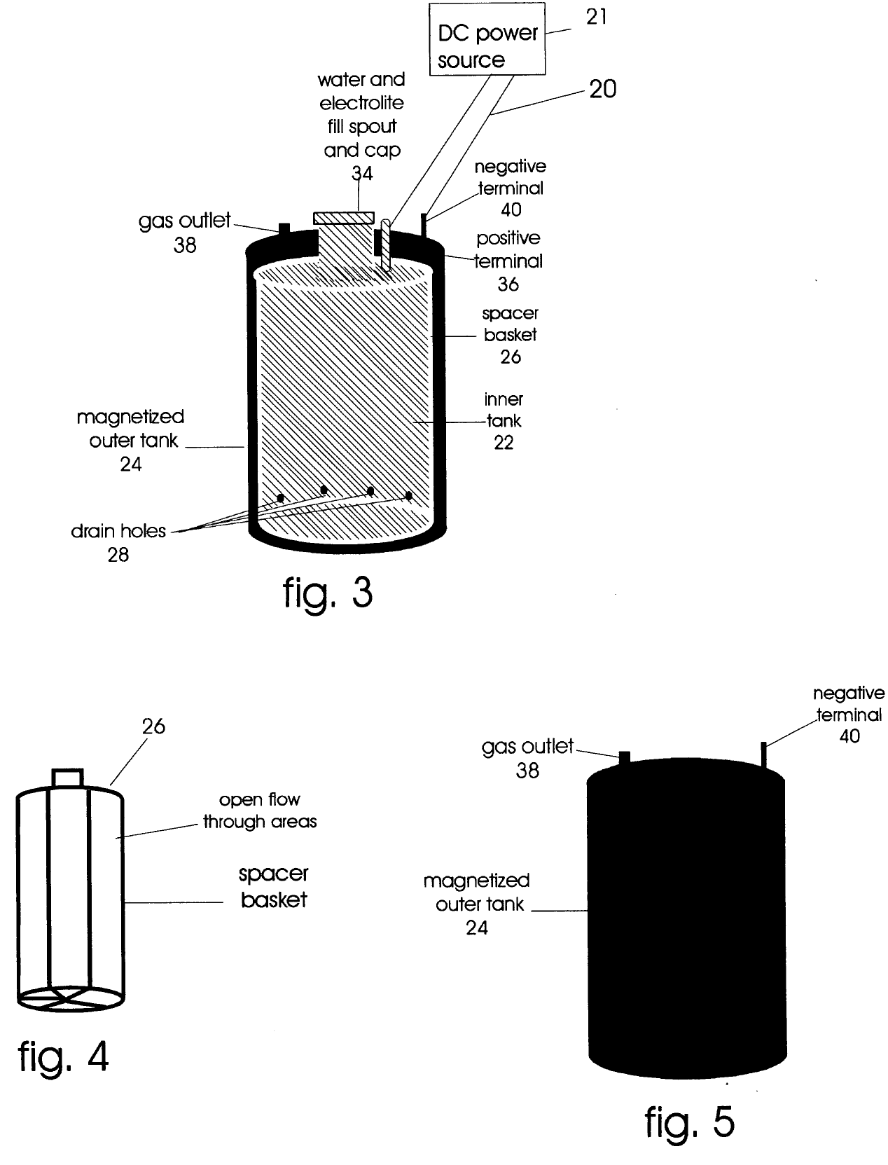

The electrolyzer that causes this reconfiguration of a water molecule is depicted in FIGS. 3-5. Electrolyzer 20 includes inner tank 22 that serves as an electrode and magnetized outer tank 24 that serves as an electrode. Spacer basket 26 is positioned between said inner and outer tanks. The distance between the inner and outer tanks controls the voltage and the volume of gas output is determined by the amperage.

Multiple drain holes, collectively denoted 28, are formed in the cylindrical sidewalls 30 of inner tank 24, near imperforate bottom wall 32 thereof. Drain holes 28 allow electrolyte in inner tank 22 to flow into the open space between the inner and outer tanks.

Inner tank 22 includes water and electrolyte fill spout 34 which is insulated from but sealed to outer tank 24 by spacer basket 26.

Positive terminal 36 is in electrical communication with the positive side of a DC power source 21. Positive terminal 36 is attached to inner tank 22 and insulated from outer tank 24.

Gas outlet 38 is attached to outer tank 24. The open end thereof may be attached to a filter, dryer, or storage tank, depending upon the application.

Negative terminal 40 is attached to outer tank 24 and the negative side of said DC power source.

Spacer basket 26 is made of a plastic or other material suitable as an electrical insulator and sufficiently resistant to withstand strong electrolytic solutions such as potassium hydroxide. It has open flow through areas as depicted in FIG. 4.

Inner tank 22 and outer tank 24 are made of material capable of being magnetized.

Outer tank 24 is magnetized by a permanent magnet or an electromagnet with sufficient Gauss units to affect inner tank 22. In a commercial embodiment, the strength of the magnetic field is about twenty (20) Gauss units.

The properties of the novel polarized gas are not limited to increase in flame temperature property mentioned above. Bubbling the novel polarized flammable gas through distilled water results in bonding with many organic and inorganic substances and rendering them harmless. Even distilled water, filtered water, and reverse osmosis water still contain free radicals and traces of bacteria. Lab tests have shown that the water produced by bubbling the novel polarized gas through it is more conductive, non-toxic, and bacteria free. Controlled studies on plants have shown that it makes them healthier and faster-growing. Wounds heal faster on mammals cleaned daily with the novel water. People who have imbibed the water anecdotally recount the stopping of migraine headaches, clearer vision, improved concentration, and other improvements such as dissolving vitamins, minerals and nutrients faster.

Without regard to what is eaten or what kind of medicine, vitamins, or supplements are taken, if the body does not metabolize it, it doesn't provide any benefits. Metabolism is the sum of all chemical changes that take place in the body. These changes are responsible for maintaining health and providing energy, but energy is also required to bring about these changes. Since water is the liquid the body uses to dissolve and transport foods, vitamins, nutrients, and medicines, it is essential to energize this vital fluid in order for the body to function properly and to heal itself.

When water evaporates from the earth, it rises through a powerful magnetic field that induces a charge. Lightning is the discharge of this stored energy. As rain drops fall back to earth, a small amount of energy is also induced which cleans the air, neutralizes pollution, and has enough energy left over to make lawns greener, healthier, and grow three (3) times faster than they would grow with ordinary water. Even the slightest polarization of water, as evidenced by rain, is extremely beneficial in nature. In order to control the degree of polarization, the polarized gas is made from water as disclosed above and then bubbled through a quantity of distilled water as aforesaid.

The novel gas produced by the novel electrolyzer thus enables the production of a novel water having numerous therapeutic effects. The novel gas has many other applications as well, such as the enabling of more efficient combustion of fuels, and so on, all of which are inherent and thus within the scope of this invention.

It will thus be seen that the objects set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention that, as a matter of language, might be said to fall therebetween.

Now that the invention has been described,

What is claimed is:

1. An electrolyzer, comprising:
a first electrode in the form of an inner tank having a first diameter formed of a material capable of being magnetized;
a second electrode in the form of an outer tank having a second diameter formed of a material capable of being magnetized;
said second diameter being greater than said first diameter;
said inner tank being nested within said outer tank;
a cylindrical open space created between said inner and outer tanks by said difference in diameters;
said inner tank having drain holes formed therein so that an electrolytic liquid fluid is disposed in said open space between said inner tank and said outer tank when said inner tank is filled with said electrolytic fluid;
a spacer basket formed of a plastic material disposed in said open space between said inner tank and said outer tank;
a DC power source;
a positive terminal secured to said inner tank, said positive terminal in electrical communication with a positive side of said DC power source;
a negative terminal secured to said outer tank, said negative terminal in electrical communication with a negative side of said DC power source; and
a magnet for magnetizing said outer tank, said magnet having a field sufficient to affect said inner tank.

2. The electrolyzer of claim 1, further comprising:
said DC power source providing a voltage of about 1.5 to 2.0 volts and a current of about 0.25 amps per square inch of cathode surface area.

3. The electrolyzer of claim 1, further comprising:
said magnetic field having a strength of about twenty Gauss units.

4. The electrolyzer of claim 1, further comprising:
said electrolytic fluid being potassium hydroxide.

5. The electrolyzer of claim 1, further comprising:
said outer tank having a gas outlet;
a flammable gas being generated by electrolysis within said electrolyzer and said flammable gas flowing through said gas outlet;
said flammable gas burning at a temperature of about two hundred seventy degrees Fahrenheit (270° F.) when ignited;
said flammable gas, when burning in contact with a substance, having a flame temperature substantially equal to a melting temperature of said substance.

6. The electrolyzer of claim 5, further comprising:
said flammable gas having a first hydrogen atom bonded with an oxygen atom and a second hydrogen atom bonded with said first hydrogen atom.

7. The electrolyzer of claim 6, further comprising:
a purified, polarized water made by passing said flammable gas through distilled water.
